SEQUENCE LISTING (4 sequences)

SEQ ID NO: 1 - DNA, 761 bp, synthetic construct
  source 1..761: mol_type = other DNA; note = Debaryomyces hansenii - ITS Forward primer
nnnnnnnnnnnnnnngtangtgacctgcggaggatcattacagtattctttttgccagcgcttaattgcgcggcgaaaaaaccttacacacagtgttttttgttattacaagaacttttgctttggtctggactagaaatagtttgggccagaggtttactgaactaaacttcaatatttatattgaattgttatttatttaattgtcaatttgttgattaaattcaaaaaatcttcaaaactttcaacaacggatctcttggttctcgcatcgatgaagaacgcagcgaaatgcgataagtaatatgaattgcagattttcgtgaatcatcgaatctttgaacgcacattgcgccctctggtattccagagggcatgcctgtttgagcgtcatttctctctcaaaccttcgggtttggtattgagtgatactcttagttgaactaggcgtttgcttgaaatgtattggcatgagtggtactggatagtgctatatgactttcaatgtattaggtttatccaactcgttgaatagtttaatggtatatttctcggtattctaggctcggccttacaatataacaaacaagtttgacctcaaatcaggtaggattacccgctgaacttaagcatatcantaannnggnaggaannnnnnnnnnnnngnnttnnnnnnnngngggggggnnnnnngggngnngntgnnnnnnnnnnggnnnnnnnnnnnnnnnnnnntgngnnnnnnnggggngnnnngngtg

SEQ ID NO: 2 - DNA, 643 bp, synthetic construct
  source 1..643: mol_type = other DNA; note = Debaryomyces hansenii - ITS Reverse primer
nnnnnnnnnnnnnnnnnngnnttgaggtcaacttgtttgttatattgtaaggccgagcctagaataccgagaaatataccattaaactattcaacgagttggataaacctaatacattgaaagtcatatagcactatccagtaccactcatgccaatacatttcaagcaaacgcctagttcaactaagagtatcactcaataccaaacccgaaggtttgagagagaaatgacgctcaaacaggcatgccctctggaataccagagggcgcaatgtgcgttcaaagattcgatgattcacgaaaatctgcaattcatattacttatcgcatttcgctgcgttcttcatcgatgcgagaaccaagagatccgttgttgaaagttttgaagattttttgaatttaatcaacaaattgacaattaaataaataacaattcaatataaatattgaagtttagttcagtaaacctctggcccaaactatttctagtccagaccaaagcaaaagttcttgtaataacaaaaaacactgtgtgtaaggttttttcgccgcgcaattaagcgctggcaaaaagaatactgtaatgatccttccgcaggttcacctacggaaaccttgttacgacttttacttcctctaannn

SEQ ID NO: 3 - DNA, 635 bp, synthetic construct
  source 1..635: mol_type = other DNA; note = Rhodotorula mucilaginosa - ITS Forward primer
nnnnnnnnnnnngnnnnnntaggtgacctgcggaggatcattagtgaatataggacgtccaacttaacttggagtccgaactctcactttctaaccctgtgcatttgtttgggatagtaactctcgcaagagggcgaactcctattcacttataaacacaaagtctatgaatgtattaaattttataacaaaataaaactttcaacaacggatctcttggctctcgcatcgatgaagaacgcagcgaaatgcgataagtaatgtgaattgcagaattcagtgaatcatcgaatctttgaacgcaccttgcgctccatggtattccgtggagcatgcctgtttgagtgtcatgaatacttcaaccctcctctttcttaatgattgaagaggtgtttggtttctgagcgctgctggcctttacggtctagctcgttcgtaatgcattagcatccgcaatcgaacttcggattgacttggcgtaatagactattcgctgaggaattctaatcttcggattagagccgggttgggttaaaggaagcttctaatcagaatgtctacattttaagattagatctcaaatcaggtaggactacccgctgaacttannnnnnnnannggnngngnnnnnanan

SEQ ID NO: 4 - DNA, 617 bp, synthetic construct
  source 1..617: mol_type = other DNA; note = Rhodotorula mucilaginosa - ITS Reverse primer
nnnnnnnnnnnnnnnnacctganttgaganctaatcttanatgtagacnttctgattagaagcttcctttaacccaacccggctctaatccgaagattagaattcctcagcgaatagtctattacgccaagtcaatccgaagttcgattgcggatgctaatgcattacgaacgagctagaccgtaaaggccagcagcgctcagaaaccaaacacctcttcaatcattaagaaagaggagggttgaagtattcatgacactcaaacaggcatgctccacggaataccatggagcgcaaggtgcgttcaaagattcgatgattcactgaattctgcaattcacattacttatcgcatttcgctgcgttcttcatcgatgcgagagccaagagatccgttgttgaaagttttattttgttataaaatttaatacattcatagactttgtgtttataagtgaaataggagttcgccctcttgcgagagttactatcccaaacaaatgcacagggttagaaagtgagagttcggactccaagttaagttggacgtcctatattcactaatgatccttccgcaggttcacctacggaaaccttgttacgacttttactnnnnn

(19) United States
(12) Patent Application Publication
(10) Pub. No.: US 2023/0406743 A1
(43) Pub. Date: Dec. 21, 2023

(54) COMPOSITIONS

(71) Applicant: Cemvita Factory, Inc., Houston, TX (US)

(72) Inventors: Marcio Luis Busi DA SILVA, Houston, TX (US); Renata Amanda GONÇALVES, Houston, TX (US); Thomas R. CLARK, Houston, TX (US); Marina NADAL, Houston, TX (US); Mary REAKES, Houston, TX (US)

(21) Appl. No.: 18/325,614

(22) Filed: May 30, 2023

Related U.S. Application Data
(60) Provisional application No. 63/365,508, filed on May 31, 2022.

(51) Int. Cl. C02F 3/34

(52) U.S. Cl. CPC C02F 3/347; C02F 2103/10

(57) ABSTRACT

A bioremediation method comprising: providing an alkalinizing acidophilic fungus, contacting an acidic liquid having a pH of 5 or lower with the fungus, and maintaining the acidic liquid under conditions sufficient to permit the fungus to increase the pH of the acidic liquid.

This application claims priority to U.S. Provisional Patent Application No. 63/365,508, filed May 31, 2022.

### FIELD

The present disclosure relates to bioremediation systems and methods for wastewater treatment, including the mining industry, as well as organisms and compositions which may be utilized in such systems and methods.

### BACKGROUND

The treatment of contaminated wastewater from heavy industry is a major environmental concern. One example of wastewater requiring treatment is acid mine drainage (AMD) from mining operations. As the name suggests, AMD is acidic liquid which drains from mines or other facilities in which mined material is treated. The low pH of AMD has an adverse effect on aquatic life in waterways. AMD typically further comprises metals such as copper, cadmium, lead, nickel, zinc, aluminum, arsenic, iron, as well as lanthanides which exacerbate the negative impact on aquatic life. Acidic drainage can also originate from non-mined environments and in some instances, may arise naturally, but still poses similar environmental concerns as AMD. Acidic drainage originating from non-mined environments is known as acid rock drainage (ARD).

The addition of alkaline minerals to acidic drainage has been deployed to increase the pH of the acidic drainage. For example, lime addition is the most common method of treatment for acidic wastewater, and though proven effective, it is expensive in the long term, has a high carbon footprint, and produces high volumes of sludge that need further treatment for disposal. Electrochemical reactions can increase the pH of acidic wastewater and can remove some soluble heavy metals from solution by precipitation.

Bioremediation presents another alternative, with potential for a cost effective and environmentally sustainable approach to treat wastewater and other contamination resulting from mining activities. Bioremediation is a process that uses biological organisms or materials, e.g., microorganisms, plants, or microbial or plant enzymes to detoxify contaminants in environments such as water or soil. Microorganisms such as bacteria or cyanobacteria can be utilized to remove heavy metals and to increase pH levels of acidic effluents. However, many microorganisms may not be able to thrive in wastewater effluents with very low pH, or high concentrations of toxic heavy metals and thus be unsuitable for bioremediation in such environments. Sulfate-reducing bacteria have been known for their potential to neutralize pH and remove heavy metals from aqueous environments. However, the bioprocess is accompanied by the production of highly corrosive and toxic hydrogen sulfide, an unwanted byproduct which itself can cause environmental damage. Currently available methods that can treat large volumes of wastewater, such as effluents generated at mining sites, are very expensive and may lack efficiency. A need remains for wastewater treatments that are robust, cost effective, and accessible for use on an industrial scale.

There remains a need for systems and methods for wastewater treatment that can provide for effective, robust, practical and cost-effective pH adjustment and reduction of heavy metals in wastewater on an industrial scale.

### SUMMARY

Thus, according to a first aspect of the present application, there is provided a bioremediation method comprising: a. providing an alkalinizing acidophilic fungus; b. contacting an acidic liquid having a pH of 5 or lower with the alkalinizing acidophilic fungus; and c. maintaining the acidic liquid under conditions sufficient to permit the alkalinizing acidophilic fungus to increase the pH of the acidic liquid.

The inventors have surprisingly identified that acidophile fungi are able to alkalinize liquids under highly acidic conditions. This activity is particularly unexpected in AMD and ARD liquids which have high sulphate concentrations and/or significant levels of dissolved metals, which are known to be toxic to many microorganisms. Through the use of such fungi, the use of energy intensive techniques such as lime addition can advantageously be avoided.

### BRIEF DESCRIPTION OF THE DRAWINGS

FIG. 1 demonstrates the growth of both fungi continued during the course of the experiment at low pH in 0 h, 24 h, 48 h and 72 h.

### DETAILED DESCRIPTION

For the avoidance of doubt, as used herein, acidophilic is to be interpreted broadly and encompasses fungi which grow effectively in highly acidic environments as well as aciditolerant fungi which may grow equally well or less well in highly acidic environments but which nevertheless are capable of survival and/or growth in such environments while continuing to be alkalinizing.

While the occurrence and roles of fungi and algae in acid mine drainage was considered by Kanti Das et al. Water Research, Volume 43, 2009, pages 883 to 894, it was stated in that document that fungi are sensitive and that the low pH of AMD does not favor fungal growth. The use of alkalinizing fungi to increase the pH of acidic media such as AMD is therefore not disclosed or advocated in that document and is actually discouraged by it. The use of fungi in other water treatment methods has been disclosed in other documents (e.g. in Chinese patent application nos. CN111394260, CN113881582, CN113862163 and CN114381377). However, none of these applications provide an enabled disclosure of a process for increasing the pH of acidic liquid using alkalinizing fungi.

The fungus employed in the present application may be a yeast or a mold, for example a filamentous fungus or a dimorphic fungus. In embodiments, the fungus comprises one or more strains of alkalinizing yeast and/or one or more strains of alkalinizing mold. In certain embodiments of the invention, one or more strains of alkalinizing yeast and/or one or more strains of alkalinizing mold may be contacted with the acidic liquid.

The fungus employed is alkalinizing, i.e. it is able to increase the pH of liquids with which it is contacted. In embodiments of the invention, fungus may be employed which is able to alkalinize fluids via any mechanism. In a preferred embodiment, the fungus is an ammonia producing fungus.

The acidic liquid, prior to being contacted with the alkalinizing acidophilic fungus, may have a pH of 5 or lower, 4 or lower, 3 or lower or 2 or lower.

In embodiments of the invention, the concentration of cells of the alkalinizing acidophilic fungus in the acidic liquid following contacting of the fungus with the acidic liquid may be at least about 1×10³ CFU/mL, at least about 1×10⁴ CFU/mL, at least about 1×10⁵ CFU/mL, at least about 1×10⁷ CFU/mL, at least about 1×10⁸ CFU/mL, at least about 1×10⁹ CFU/mL, at least about 1×10¹⁰ CFU/mL, at least about 1×10¹¹ CFU/mL, at least about 1×10¹² CFU/mL or at least about 1×10¹³ CFU/mL.

The acidic liquid may additionally comprise dissolved metal/s, for example, dissolved copper, iron, nickel, cadmium, strontium, mercury, lead, arsenic, aluminum, lithium, zinc, manganese, lanthanides and/or others. Advantageously, in embodiments in which the acidic liquid comprises dissolved metals, the increase in pH caused by the alkalinizing acidophilic fungus can result in the precipitation of metals from the acidic liquid. In such embodiments, the metal may be precipitated in any form, e.g., as metal (in its original valence state or in an altered valence state) and/or in the form of one or more salts. Thus, in embodiments of the invention, the process further comprises the step of collecting metal precipitated from the acidic liquid.

The acidic liquid may comprise anions, for example sulphate ions, at a concentration of at least about 0.01 grams per liter, at least about 0.02 grams per liter, at least about 0.05 grams per liter, at least about 0.1 grams per liter, at least about 0.2 grams per liter, at least about 0.5 grams per liter or at least about 1 gram per liter. Additionally or alternatively the acidic liquid may comprise sulphate ions at a concentration of about 100 grams per liter or less, about 50 grams per liter or less, about 20 grams per liter or less, or about 10 grams per liter or less.

The process may additionally comprise the addition of a nutrition source to the acidic liquid, for example a nitrogen source such as an amino acid or protein source (e.g. soybean residues, effluent from the dairy industry, or any other amino acid and/or protein-rich wastewater) and/or a carbon source (e.g. molasses). The nutrition source may be comprised within a composition comprising the alkalinizing acidophilic fungus and/or be separately added to the acidic liquid. In some embodiments a plurality of nutrition sources may be added to the acidic liquid, for example a first nutrition source (which may or may not be comprised within a composition comprising the alkalinizing acidophilic fungus) and a second nutrition source.

In embodiments of the invention, the pH of the acidic liquid may be increased following contact with the alkalinizing acidophilic fungus to 5 or higher, 6 or higher, 7 or higher, 8 or higher or 9 or higher.

The alkalinizing acidophilic fungus may be provided in a composition of any form known to those skilled in the art. In such embodiments, the composition comprising the alkalinizing acidophilic fungus may be an inoculum, spores, a lyophilizate, a liquid concentrate, a fungal cell suspension (e.g. a planktonic type cell culture), or immobilized cells (e.g. where the cells are encapsulated with alginate) or a combination thereof. Thus, according to a further aspect of the present application, there is provided a composition comprising an alkalinizing acidophilic fungus.

The alkalinizing acidophilic fungus may comprise a strain which optionally belongs to the following genera: Bullera, Cadophora, Debaromyces, Filobasidium, Leucosporidium, Naganishia, Penicillium, Rhodotorula, Solicoccozyma, Acontium, Aspergillus, Aureobasidium, Cephalosporium, Cladosporium, Cryptococcus, Fusarium, Geotrichum, Mucor, Zygorhynchus, Trichoderma, Phoma, Saccharomyces, Scytalidium, Aureobasidium, Filobasidium, Hannaella, Candida, Auriculibuller, Papiliotrema, Pseudozyma, Hannaella, Microbotryozyma, Meyerozyma or a combination thereof. In specific embodiments the alkalinizing acidophilic fungus is a yeast belonging to either the Debaromyces or Rhodotorula genera, preferably one belonging to the species Debaryomyces hansenii and/or Rhodotorula mucilaginosa. In certain embodiments, the alkalinizing acidophilic fungus does not belong to the Aspergillus, Paecilomyces and/or Penicillium genera or the Aspergillus koji or Rhodotorula taiwanensis species. In some embodiments, the alkalinizing acidophilic fungus does not belong to the strain Rhodotorula taiwanensis MF4, Aspergillus koji MF1, or Penicillium MF2 or MF3

In embodiments, the alkalinizing acidophilic fungus may comprise a mold (e.g. a dimorphic fungus and/or a filamentous fungus).

The fungus may comprise a single strain of fungus. Alternatively, the fungus may comprise a consortium of fungal strains, for example, comprising 2 or more, 3 or more, 4 or more, 5 or more strains of fungus. In embodiments of the invention, the fungus comprises one or more strains of yeast. Additionally, or alternatively, the fungus comprises one or more strains of mold (e.g. one or more strains of filamentous fungi and/or one or more strains of dimorphic fungi)

Additionally, or alternatively, the alkalinizing acidophilic fungus may comprise a psychrophile. For example, the fungus may be able to reproduce and/or alkalinize liquids at temperatures of 20° C. or lower, 15° C. or lower, 10° C. or lower or 5° C. or lower.

In embodiments of the invention, the fungus may be native, i.e. it may be non-engineered. In alternative embodiments, the fungus may be engineered to introduce or enhance its phenotypic properties to optimize it for use in the present application.

In embodiments of the invention, the process may be conducted in a bioreactor, for example a device that includes of one or more vessels and/or towers or piping arrangement, which includes the Batch Reactor, Continuous Stirred Tank Reactor (CSTR), Immobilized Cell Reactor (ICR), Trickle Bed Reactor (TBR), Bubble Column, Gas lift Fermenter, Static Mixer, or other device suitable for gas-liquid contact. One example of such a bioreactor which is suitable for use in the present application is that disclosed in U.S. Ser. No. 63/248,141, the contents of which are incorporated herein by reference.

In embodiments in which the process of the invention is conducted in a bioreactor, the acidic liquid may be added into the bioreactor. A nutrition source may also be added, e.g. a carbon source (such as molasses) and/or a nitrogen source. Additionally, the alkalinizing acidophilic fungus may be added, e.g. in the form of an inoculum. In preferred embodiments of the invention, the concentration of fungal cells present in the acidic liquid is 1×10⁷ CFU/mL to about 1×10¹³ CFU/mL.

In embodiments of the invention, the bioreactor may be a batch reactor. In such embodiments, operation of the batch reactor may be discontinued once the pH and/or precipitated reach target values. Following discontinuation of operation of the reactor, 50% of the acidic liquid may be removed from the reactor, and a corresponding quantity of acidic liquid, optionally comprising a nutrition source (e.g. a nitrogen source and/or a carbon source) may be charged into the reactor.

In embodiments of the invention, the bioreactor may be a continuously operated reactor, e.g. a flow-type reactor. In such embodiments, the acidic medium may be continuously fed into the reactor. Fungus and optionally a nutrition source may also be fed into the reactor continuously and/or intermittently. The retention time of the acidic liquid in the reactor may be calculated based on the overall volume of reactor and growth rate of the fungus.

In certain embodiments, the recirculation through continuous reactors, such as flow-type reactors may be up to 30% or up to 50%. In other embodiments, recirculation through continuous reactors may be 10 to 20%, 20 to 30%, 30 to 40% or 40 to 50%. The skilled reader will be familiar with calculating recirculation levels in continuous reactors based on reactor input and output flow as well as hydraulic retention times.

In certain embodiments, fungal cells may be immobilized whether the process is operated in a reactor or in situ. Any suitable material may be used as an immobilization matrix. In embodiments, the immobilization matrix may be configured to permit attachment of the fungal cells and/or the growth of biofilms thereupon.

In embodiments of the invention, the fungus may be provided in the form of suspended cells, for example, a planktonic type cell culture or immobilized cells, for example, encapsulated with alginate. Providing the fungus in the form of immobilized cells protects the cells against harsh conditions, for example extreme low pH and or inhibitory metals concentrations.

In embodiments, the process may be conducted in situ, i.e. without transferring the acidic liquid into a purpose-built bioreactor but working the process in the environment in which the acidic liquid is present or transferring it to a natural environment in which the process can be worked, e.g. wells or subsurface caverns. While certain remediation processes cannot be worked in situ because of the use or production of environmentally damaging compounds, an advantage of the present application is that the alkalizing fungi employed therein are ecologically benign and are unlikely to cause environmental damage. In embodiments of the invention, to increase the alkalinizing efficiency of the fungus, techniques may be employed with which the skilled person will be familiar, such as the use of injection wells, pump and treat techniques, and/or the provision of oxygen source/s.

As explained herein, the process of the present application is carried out on an acidic liquid. The acidic liquid may be derived from any source or industrial process. In embodiments, the acidic liquid may be wastewater, e.g. from mining operations (such as AMD), from petrochemical production, from e-waste treatment, or from mine tailings. In other embodiments, the acidic liquid may be naturally occurring, e.g. ARD.

The disclosure provided herein will be better understood when read in conjunction with the attached drawings. It should be understood that where certain embodiments may be described as being preferable, they should not be considered limiting and may be combined.

Herein incorporated by reference is the sequence listing filed with the USPTO as P12909W0.xml which was created on May 26, 2023, and the size is 7,908 bytes.

Unless otherwise noted, all instances of the words “a,” “an,” or “the” can refer to ‘one’ or ‘more than one of’.

Unless otherwise noted, the terms “heavy metal” or “heavy metals” refers to. copper, cadmium, lead, nickel, zinc, aluminum, arsenic, iron, and lanthanides.

Unless otherwise noted, where used to describe a strain of fungus, the term “engineered” refers to non-native fungal strains that are a product of genetic manipulation. In some embodiments, engineered fungal strains may comprise non-native genes. Additionally or alternatively, in some embodiments, engineered fungal strains may over-express native genes.

Acidophiles are defined as organisms which are found in acidic environments and grow optimally at pH<6.

18S gene sequencing may be used to assist with the taxonomic identification of fungal strains. Methods to determine sequence identity and similarity (e.g. PCR amplification and sequencing of yeast 26S ribosomal RNA (rRNA) and Internal Transcribed Spacer 1 and 2 regions (ITS1 & ITS2)) provides organism identity and similarity) are codified in publicly available computer programs. Exemplary computer program methods to determine identity and similarity between two sequences include e.g., the BestFit, BLASTP (Protein Basic Local Alignment Search Tool), BLASTN (Nucleotide Basic Local Alignment Search Tool), and FASTA (Altschul, S. F. et al., J. Mol. Biol. 215:403-410 (1990), publicly available from NCBI 25 and other sources (BLAST.RTM. Manual, Altschul, S., et al., NCBI NLM NIH Bethesda, Md. 20894). A most exemplary algorithm used is EMBOSS (European Molecular Biology Open Software Suite). Exemplary parameters for amino acid sequences comparison using EMBOSS are gap open 10.0, gap extend 0.5, BLOSUM matrix. Exemplary parameters for nucleic acid sequences comparison using EMBOSS are gap open 10.0, gap extend 0.5, DNA full matrix 30 (DNA identity matrix). In embodiments, it is possible to compare the DNA/protein sequences among different species to determine the homology of sequences using online data such as Gene bank, KEGG, BLAST and Ensemble.

Embodiments of the present application can provide the benefit of removing heavy metals from acidic liquids such as wastewater in a robust, efficient, and cost-effective manner. Such embodiments can also provide a benefit of raising the pH of acidic liquids such as wastewater to environmentally acceptable levels. Embodiments of the invention discussed herein can also provide a benefit of the removal of heavy metals from aqueous liquids on an industrial scale.

Embodiments herein employ one or more strains of alkalinizing acidophilic fungus, which can provide an eco-friendly alternative treatment to remove heavy metals from acidic liquids such as wastewater.

As demonstrated in the accompanying examples, acid-neutralizing fungus can be used as bio-machinery for ammonia production. Excreted ammonia increases pH in an acidic liquid, for example mining acid wastewater. This process may also result in the precipitation of metals.

Embodiments herein are directed to methods for the treatment of acidic liquids. In various embodiments, the method comprises providing an alkalinizing acidophilic fungus, contacting an acidic liquid having a pH of 5 or lower with the fungus, and maintaining the acidic liquid under conditions sufficient to permit the fungus to increase the pH of the acidic liquid to neutrality.

In certain embodiments, the acidic liquid, prior to being contacted with the fungus, has a pH of 5 or lower, 4 or lower, 3 or lower, or 2 or lower.

In embodiments of the invention, a fungus may be employed which has the ability to alkalinize liquids under highly acidic conditions. The fungus employed can increase the pH of the acidic liquid. Such fungus may be employed to alkalinize fluids via any mechanism. In certain embodiments, the fungus may be ammonia producing fungus. In preferred embodiments of the invention, the fungus is an ammonia producing fungus. Additionally or alternatively, the fungus may cause precipitation of the metal via complexation caused by pH increase.

In certain embodiments, the acidic liquid may additionally comprise dissolved metal/s. The dissolved metals may include one or more of dissolved copper, iron, nickel, cadmium, strontium, mercury, lead, arsenic, aluminum, lithium, zinc, lanthanides and/or manganese, or others. In certain embodiments, an increase in pH can result in precipitation of metals from the acidic liquid, e.g. in the form of metal per se (in its original valence state or in an altered valence state) and/or in the form of a salt. In certain embodiments, the method further comprises collecting metal precipitated from the acidic liquid.

In embodiments of the invention the acidic liquid may comprise dissolved heavy metals at a concentration of about 1 mg per liter or more, about 2 mg per liter or more, about 5 mg per liter or more, about 10 mg per liter or more, about 20 mg per liter or more, about 50 mg per liter or more, about 100 mg per liter or more, about 200 mg per liter or more, about 500 mg per liter or more, or about 1000 mg per liter or more.

Additionally, or alternatively, the acidic liquid may comprise one or more of the following dissolved metals:
Iron, optionally at a concentration of about 0.1 mg per liter or more, about 0.2 mg per liter or more, about 0.5 mg per liter or more, about 1 mg per liter or more, about 2 mg per liter or more, about 5 mg per liter or more, about 10 mg per liter or more, about 20 mg per liter or more, about 50 mg per liter or more, about 100 mg per liter or more, about 200 mg per liter or more or about 500 mg per liter or more and/or 1000 mg per liter or less;
Manganese, optionally at a concentration of about 0.1 mg per liter or more, about per liter or more, about 0.5 mg per liter or more, about 1 mg per liter or more, about 2 mg per liter or more, about 5 mg per liter or more, about 10 mg per liter or more, about 20 mg per liter or more, about 50 mg per liter or more, about 100 mg per liter or more, about 200 mg per liter or more or about 500 mg per liter or more;
Copper, optionally at a concentration of about 0.1 mg per liter or more, about per liter or more, about 0.5 mg per liter or more, about 1 mg per liter or more, about 2 mg per liter or more, about 5 mg per liter or more, about 10 mg per liter or more, about 20 mg per liter or more, about 50 mg per liter or more, about 100 mg per liter or more, about 200 mg per liter or more or about 500 mg per liter or more;
Zinc, optionally at a concentration of about 0.01 mg per liter or more, about per liter or more, about 0.05 mg per liter or more, about 0.1 mg per liter or more, about per liter or more, about 0.5 mg per liter or more, about 1 mg per liter or more, about 2 mg per liter or more, about 5 mg per liter or more, about 10 mg per liter or more, about 20 mg per liter or more, about 50 mg per liter or more, about 100 mg per liter or more, about 200 mg per liter or more, about 500 mg per liter or more or about 1,000 mg per liter or more;
Nickel, optionally at a concentration of about 0.01 mg per liter or more, about per liter or more, about 0.05 mg per liter or more, about 0.1 mg per liter or more, about per liter or more, about 0.5 mg per liter or more, about 1 mg per liter or more, about 2 mg per liter or more, about 5 mg per liter or more, about 10 mg per liter or more, about 20 mg per liter or more or about 50 mg per liter or more;
Cobalt, optionally at a concentration of about 0.01 mg per liter or more, about per liter or more, about 0.05 mg per liter or more, about 0.1 mg per liter or more, about per liter or more, about 0.5 mg per liter or more, about 1 mg per liter or more, about 2 mg per liter or more, about 5 mg per liter or more, about 10 mg per liter or more, about 20 mg per liter or more or about 50 mg per liter or more;
Arsenic, optionally at a concentration of about 0.01 mg per liter or more, about per liter or more, about 0.05 mg per liter or more, about 0.1 mg per liter or more, about per liter or more, about 0.5 mg per liter or more, about 1 mg per liter or more, about 2 mg per liter or more, about 5 mg per liter or more, about 10 mg per liter or more, about 20 mg per liter or more or about 50 mg per liter or more;
Cadmium, optionally at a concentration of about 0.01 mg per liter or more, about per liter or more, about 0.05 mg per liter or more, about 0.1 mg per liter or more, about per liter or more, about 0.5 mg per liter or more, about 1 mg per liter or more, about 2 mg per liter or more, about 5 mg per liter or more, about 10 mg per liter or more, about 20 mg per liter or more, about 30 mg or more or about 50 mg per liter or more and/or about 35 mg per liter or less;
Lead, optionally at a concentration of about 0.01 mg per liter or more, about per liter or more, about 0.05 mg per liter or more, about 0.1 mg per liter or more, about per liter or more, about 0.5 mg per liter or more, about 1 mg per liter or more, about 2 mg per liter or more, about 5 mg per liter or more, about 10 mg per liter or more, about 20 mg per liter or more or about 50 mg per liter or more;
Aluminum, optionally at a concentration of about 0.01 mg per liter or more, about per liter or more, about 0.05 mg per liter or more, about 0.1 mg per liter or more, about per liter or more, about 0.5 mg per liter or more, about 1 mg per liter or more, about 2 mg per liter or more, about 5 mg per liter or more, about 10 mg per liter or more, about 20 mg per liter or more, about 50 mg per liter or more, about 100 mg per liter or more, about 200 mg per liter or more, about 500 mg per liter or more, about 750 mg per liter or more and/or about 1300 mg per liter or less; or
Lanthanides, optionally at a concentration of about 0.01 mg per liter or more, about 0.02 mg per liter or more, about 0.05 mg per liter or more, about 0.1 mg per liter or more, about 0.2 mg per liter or more, about 0.5 mg per liter or more, about 1 mg per liter or more, about 2 mg per liter or more, about 5 mg per liter or more, about 10 mg per liter or more, about 20 mg per liter or more or about 50 mg per liter or more.

In certain embodiments, the acidic liquid may comprise sulphate ions at a concentration ranging from 0.1 to 20 grams per liter (g/L). The acid liquid may also comprise sulphate ions at a concentration ranging from 0.1 to 5 g/L, 5 to 10 g/L, 10 to 15 g/L and 15 to 20 g/L.

Advantageously, the process of the present application may be conducted on an industrial scale. For example, in embodiments of the invention, the acidic liquid has a volume of about 10 or more liters, about 20 or more liters, about 50 or more liters, about 100 or more liters, about 200 or more liters, about 500 or more liters, about 1,000 or more liters, about 2,000 or more liters, about 5,000 or more liters, about 10,000 or more liters, about 20,000 or more liters, about 50,000 or more liters, about 100,000 or more liters, about 200,000 or more liters or about 500,000 or more liters.

It has been found that the processes of the present application can be operated at the pH of the acidic liquid, i.e. without a pre-treatment pH adjustment step being performed or a buffer being used. Thus, in embodiments of the invention, prior to the fungus being contacted with the acidic liquid, no pH adjustment step is performed. In such, or alternative, embodiments, the acidic liquid is not buffered, for example, no buffer is added to the acidic liquid.

In certain embodiments, a nutrition source is added to the acidic liquid. In such embodiments, the nutrition source may be added prior to, simultaneous with, or following the step of contacting the acidic medium with the fungus. Preferably the nutrition source is a nitrogen source, such as an amino acid and/or protein source, and/or a carbon source. The protein source may be soybean residues, effluent from the dairy industry, or another low nitrogen-rich wastewater rich in amino acid and/or protein. The carbon source may be molasses. In such embodiments, the nutrition source may be comprised within a composition comprising the alkalinizing acidophilic fungus and/or separately added to the acidic liquid.

In embodiments of the invention, the pH of the acidic liquid may be increased following contact with the alkalinizing acidophilic fungus to 5 or higher, or 6 or higher, 7 or higher, 8 or higher or 9 or higher. In certain embodiments, the acidic liquid, prior to being contacted with the fungus, may have a pH of 5 or lower, 4 or lower, 3 or lower or 2 or lower. In other embodiments, the acidic liquid, prior to being contacted with the fungus, may have a pH from about 2 to 3, 3 to 4, or 4 to 5. In certain embodiments, the pH of the acidic liquid is increased, through performance of the process of the present application by 2 pH units or more, by 3 pH units or more, by 4 pH units or more, by 5 pH units or more, by 6 pH units or more or by 7 pH units or more.

In certain embodiments, the alkalinizing fungus may comprise a strain optionally selected from one of the following genera: Bullera, Cadophora, Debaromyces, Filobasidium, Leucosporidium, Naganishia, Penicillium, Rhodotorula, Solicoccozyma. Acontium, Aspergillus, Aureobasidium, Cephalosporium, Cladosporium, Cryptococcus, Fusarium, Geotrichum, Mucor, Zygorhynchus, Trichoderma, Phoma, Saccharomyces, Scytalidium, Aureobasidium, Filobasidium, Hannaella, Candida, Auriculibuller, Papiliotrema, Pseudozyma, Hannaella, Microbotryozyma, Meyerozyma or a combination thereof.

In embodiments of the invention, the alkalinizing fungus employed in the process of the present application does not produce hydrogen sulphate (H2S). Additionally or alternatively, the alkalinizing fungus does not adsorb and/or sequester dissolved metals. In certain embodiments, the alkalinizing fungus is not engineered to adsorb and/or sequester dissolved metals.

The inventors have surprisingly and unexpectedly identified that alkalinizing acidophilic fungi may be heavy metal resistant, i.e. said fungi are not only capable of remaining viable in acidic liquid media comprising heavy metals dissolved in the liquid (such as ARD, AMD and other wastewater) but also of retaining their ability to grow and alkalinize the liquid. Thus, in embodiments of the invention, the alkalinizing acidophilic fungus employed in the process of the present application may be heavy metal resistant.

Heavy metal resistant strains can be identified using routine techniques with which those skilled in the art will be familiar. For example, fungal strains can be screened in samples of acidic liquid media comprising dissolved heavy metals and their growth and alkalinizing ability determined, as shown in the examples which follow.

The heavy metal resistance of the fungus may be assessed by comparing the time taken for the fungus in an acidic liquid having a given concentration of dissolved heavy metal/s (e.g. 500 mg/liter) versus that in a reference liquid which is free of dissolved heavy metal/s (with otherwise identical composition and pH, and under identical reaction conditions) to increase pH.

A fungus may be said to be heavy metal resistant if the time taken to increase the pH of the acidic liquid having the given concentration of dissolved heavy metal/s by three pH units is 2 times or less, 1.8 times or less, 1.6 times or less, 1.4 times or less or 1.2 times or less longer than the time taken to increase the pH of the reference acidic liquid which is free of dissolved heavy metal/s by three pH units.

A fungus may also be said to be heavy metal resistant if the time taken to increase the pH of the acidic liquid having the given concentration of dissolved heavy metal/s by one pH unit is 2 times or less, 1.8 times or less, 1.6 times or less, 1.4 times or less or 1.2 times or less longer than the time taken to increase the pH of the reference acidic liquid which is free of dissolved heavy metal/s by three pH units.

Additionally or alternatively, a fungus may be said to be heavy metal resistant if, when contacted with an acidic liquid medium comprising heavy metals dissolved therein (e.g. comprising the metals discussed herein and the concentrations discussed herein, such as cadmium at a level of 2 mg per liter, copper at a level of 100 mg per liter, lead at a level of 0.05 mg per liter, iron at a level of 200 mg per liter, nickel at a level of 0.2 mg per liter and/or zinc at a level of 1,000 mg per liter), it retains its alkalinizing ability for at least 24 hours of contact with the acidic liquid medium.

In certain embodiments, the fungus comprises a single strain of fungus. In other embodiments, the fungus comprises a consortium of fungal strains, optionally wherein the fungus comprises 2 or more, 3 or more, 4 or more, 5 or more, 6 or more, 7 or more, 8 or more, 9 or more or 10 or more strains of fungus. In embodiments of the invention, the fungus may comprise one or more strains of yeast. Additionally or alternatively, the fungus may comprise one or more strains of mold (e.g. one or more strains of filamentous fungi and/or one or more strains of dimorphic fungi).

In embodiments of the invention, the fungus may be comprised in a composition comprising a single strain of fungus. In other embodiments, the fungus may be comprised in a composition comprising a consortium of fungal strains, optionally wherein the composition comprises 2 or more, 3 or more, 4 or more, 5 or more, 6 or more, 7 or more, 8 or more, 9 or more or 10 or more strains of fungus. In embodiments of the invention, the composition may comprise one or more strains of yeast. Additionally or alternatively, the composition may comprise one or more strains of mold (e.g. one or more strains of filamentous fungi and/or one or more strains of dimorphic fungi)

Additionally or alternatively, in embodiments of the invention, the fungus may be comprised in a plurality of fungus-containing compositions. For example, a first fungus-containing composition may be provided and/or contacted with the acidic liquid, which first fungus-containing composition may comprise one or more strains of fungus. In embodiments of the invention, the first fungus-containing composition may comprise one or more strains of yeast. Additionally or alternatively, the first fungus-containing composition may comprise one or more strains of mold (e.g. one or more strains of filamentous fungi and/or one or more strains of dimorphic fungi).

Simultaneously, sequentially or separately, a second fungus-containing composition may be provided and/or contacted with the acidic liquid, which second fungus-containing composition may comprise one or more strains of fungus. In embodiments of the invention, the second fungus-containing composition may comprise one or more strains of yeast. Additionally or alternatively, the second fungus-containing composition may comprise one or more strains of mold (e.g. one or more strains of filamentous fungi and/or one or more strains of dimorphic fungi).

In embodiments of the invention, a single strain of fungus is contacted with the acidic liquid. In alternative embodiments of the invention, the acidic liquid is contacted with a plurality of strains of fungus, for example 1 to 50, 1 to 30, 1 to 20, 1 to 15, 1 to 10, 1 to 9, 1 to 8, 1 to 7, 1 to 6, 1 to 5, 1 to 4, 1 to 3 or 1 to 2 strains of fungus. Additionally or alternatively, the acidic liquid may be contacted with 2 or more, 3 or more, 4 or more, 5 or more, 6 or more, 7 or more, 8 or more 9 or more, or 10 or more strains of fungus. In certain embodiments, the acidic liquid may be contacted with 50 or fewer, 40 or fewer, 30 or fewer, 25 or fewer, 20 or fewer, 15 or fewer or 10 or fewer strains of fungus. In embodiments of the invention in which the fungus comprises a plurality of strains, these may be comprised within a single composition. Alternatively, they may be provided within a plurality of compositions. Where multiple strains of fungus are employed in the present application, they may comprise one or more strains of yeast and/or one or more strains of mold (e.g. one or more strains of filamentous fungi and/or one or more strains of dimorphic fungi).

In certain embodiments, the alkalinizing acidophilic fungus is a psychrophile. In certain embodiments, the psychrophilic fungus is able to reproduce and/or alkalinize liquids at temperatures of 20° C. or lower, 15° C. or lower, 10° C. or lower or 5° C. or lower.

In certain embodiments, the fungus may be native, i.e. it may be non-engineered. In alternative embodiments, the fungus may be engineered to introduce or enhance its phenotypic properties to optimize it for use in the present application.

In embodiments, the process may be conducted in situ, i.e. without transferring the acidic liquid into a purpose-built bioreactor but working the process in the environment in which the acidic liquid is present or transferring it to a natural environment in which the process can be worked, e.g. wells or subsurface caverns. In such embodiments, process controls can still be performed, for example, the metal concentration and/or pH of the aqueous medium may be altered, e.g. by the addition of fresh water.

While certain remediation processes of the prior art cannot be worked in situ because of the use or production of environmentally damaging compounds, an advantage of the present application is that the alkalinizing fungus employed therein are ecologically benign and are unlikely to cause environmental damage. In embodiments of the invention, to increase the alkalinizing efficiency of the fungus, techniques may be employed with which the skilled person will be familiar, such as the use of injection wells, pump and treat techniques, and/or the provision of oxygen source/s.

In embodiments in which the process of the invention is conducted in situ, a bioreactor may be installed in the proximity of the site in which the acidic liquid is located. In embodiments in which the acidic liquid are located below the surface (for example groundwater, in ores, in soil piles, in composting, in landfill, or other subsurface bodies of acidic liquid), the bioreactor may be located adjacent to or above the site. The fungus and optionally a nutrition source may be located within the bioreactor, for example to culture the fungus and increase the cell count. The medium within the bioreactor may then be injected (e.g. using injection wells) into the acidic liquid on a continuous or batch-wise basis. In embodiments, the acidic liquid may be fed back into the bioreactor or treated exclusively in situ. Techniques to stimulate growth or maintain viability of the fungal cells in situ may be performed, for example through the continuous injection of oxygen (for example, through the addition of hydrogen peroxide or oxygen releasing compounds), a nutrition source (i.e., reagents containing nitrogen and/or phosphorus) and/or a carbon source (for example, molasses or protein-rich diluted wastewater).

According to a further aspect of the present application, there is provided a kit comprising a composition comprising an alkalinizing acidophilic fungus and instructions for using that composition in a bioremediation process on an acidic liquid as described herein. Embodiments of the composition as employed in the bioremediation method of the present application, as described herein, also apply to the composition of the present application.

In embodiments, the instructions may be for using the composition of the invention in methods of bioaugmentation and/or biostimulation. In such embodiments, bioaugmentation and/or biostimulation may be carried out through injection wells, pump and treat, and oxygen releasing strategies, thus, maintaining fungal activity.

In certain embodiments, instructions may be for using the compositions of the invention for increasing the pH of acidic liquid such as wastewater. In certain embodiments, the composition may comprise more than one alkalinizing acidophilic fungus.

In certain embodiments, the instructions may be for using the compositions of the invention to remove metals and/or increase the pH of acidic liquid such as AMD.

The following examples are offered by way of illustration of certain embodiments of aspects of the application herein. None of the examples should be considered limiting on the scope of the application.

### EXAMPLES

### Example 1— Ability of Yeasts to Increase the pH of Acidic Liquids

Multiple strains of yeasts were identified as being capable of increasing the pH of acidic liquids while exhibiting sufficient hardiness to maintain this ability in extreme environments, such as high sulphate concentration and/or in the presence of toxic metals.

These strains belonged to the genera Bullera, Cadophora, Debaromyces, Filobasidium, Leucosporidium, Naganishia, Penicillum, Rhodotorula, and Solicococcozyma. Exemplary Debaromyces and Rhodotorula strains were identified with the following primers:

Debaryomyces hansenii

(Identity 99.4%)

ITS Forward primer
(SEQ ID NO: 1)
NNNNNNNNNNNNNNNGTANGTGACCTGCGGAGGATCATTACAGTATTCT
TTTTGCCAGCGCTTAATTGCGCGGCGAAAAAACCTTACACACAGTGTTT
TTTGTTATTACAAGAACTTTTGCTTTGGTCTGGACTAGAAATAGTTTGG
GCCAGAGGTTTACTGAACTAAACTTCAATATTTATATTGAATTGTTATT
TATTTAATTGTCAATTTGTTGATTAAATTCAAAAAATCTTCAAAACTTT
CAACAACGGATCTCTTGGTTCTCGCATCGATGAAGAACGCAGCGAAATG
CGATAAGTAATATGAATTGCAGATTTTCGTGAATCATCGAATCTTTGAA
CGCACATTGCGCCCTCTGGTATTCCAGAGGGCATGCCTGTTTGAGCGTC
ATTTCTCTCTCAAACCTTCGGGTTTGGTATTGAGTGATACTCTTAGTTG
AACTAGGCGTTTGCTTGAAATGTATTGGCATGAGTGGTACTGGATAGTG
CTATATGACTTTCAATGTATTAGGTTTATCCAACTCGTTGAATAGTTTA
ATGGTATATTTCTCGGTATTCTAGGCTCGGCCTTACAATATAACAAACA
AGTTTGACCTCAAATCAGGTAGGATTACCCGCTGAACTTAAGCATATCA
NTAANNNGGNAGGAANNNNNNNNNNNNNGNNTTNNNNNNNNGNGGGGGG
GNNNNNNGGGNGNNGNTGNNNNNNNNNNGGNNNNNNNNNNNNNNNNNNN
TGNGNNNNNNNGGGGNGNNNNGNGTG
ITS Reverse primer
(SEQ ID NO: 2)
NNNNNNNNNNNNNNNNNNGNNTTGAGGTCAACTTGTTTGTTATATTGTA
AGGCCGAGCCTAGAATACCGAGAAATATACCATTAAACTATTCAACGAG
TTGGATAAACCTAATACATTGAAAGTCATATAGCACTATCCAGTACCAC
TCATGCCAATACATTTCAAGCAAACGCCTAGTTCAACTAAGAGTATCAC
TCAATACCAAACCCGAAGGTTTGAGAGAGAAATGACGCTCAAACAGGCA
TGCCCTCTGGAATACCAGAGGGCGCAATGTGCGTTCAAAGATTCGATGA
TTCACGAAAATCTGCAATTCATATTACTTATCGCATTTCGCTGCGTTCT
TCATCGATGCGAGAACCAAGAGATCCGTTGTTGAAAGTTTTGAAGATTT
TTTGAATTTAATCAACAAATTGACAATTAAATAAATAACAATTCAATAT
AAATATTGAAGTTTAGTTCAGTAAACCTCTGGCCCAAACTATTTCTAGT
CCAGACCAAAGCAAAAGTTCTTGTAATAACAAAAAACACTGTGTGTAAG
GTTTTTTCGCCGCGCAATTAAGCGCTGGCAAAAAGAATACTGTAATGAT
CCTTCCGCAGGTTCACCTACGGAAACCTTGTTACGACTTTTACTTCCTC
TAANNN

Rhodotorula mucilaginosa

(Identity 99.7%)

ITS Forward primer
(SEQ ID NO: 3)
NNNNNNNNNNNNGNNNNNNTAGGTGACCTGCGGAGGATCATTAGTGAAT
ATAGGACGTCCAACTTAACTTGGAGTCCGAACTCTCACTTTCTAACCCT
GTGCATTTGTTTGGGATAGTAACTCTCGCAAGAGGGCGAACTCCTATTC
ACTTATAAACACAAAGTCTATGAATGTATTAAATTTTATAACAAAATAA
AACTTTCAACAACGGATCTCTTGGCTCTCGCATCGATGAAGAACGCAGC
GAAATGCGATAAGTAATGTGAATTGCAGAATTCAGTGAATCATCGAATC
TTTGAACGCACCTTGCGCTCCATGGTATTCCGTGGAGCATGCCTGTTTG
AGTGTCATGAATACTTCAACCCTCCTCTTTCTTAATGATTGAAGAGGTG
TTTGGTTTCTGAGCGCTGCTGGCCTTTACGGTCTAGCTCGTTCGTAATG
CATTAGCATCCGCAATCGAACTTCGGATTGACTTGGCGTAATAGACTAT
TCGCTGAGGAATTCTAATCTTCGGATTAGAGCCGGGTTGGGTTAAAGGA
AGCTTCTAATCAGAATGTCTACATTTTAAGATTAGATCTCAAATCAGGT
AGGACTACCCGCTGAACTTANNNNNNNNANNGGNNGNGNNNNNANAN
ITS Reverse primer
(SEQ ID NO: 4)
NNNNNNNNNNNNNNNNACCTGANTTGAGANCTAATCTTANATGTAGACN
TTCTGATTAGAAGCTTCCTTTAACCCAACCCGGCTCTAATCCGAAGATT
AGAATTCCTCAGCGAATAGTCTATTACGCCAAGTCAATCCGAAGTTCGA
TTGCGGATGCTAATGCATTACGAACGAGCTAGACCGTAAAGGCCAGCAG
CGCTCAGAAACCAAACACCTCTTCAATCATTAAGAAAGAGGAGGGTTGA
AGTATTCATGACACTCAAACAGGCATGCTCCACGGAATACCATGGAGCG
CAAGGTGCGTTCAAAGATTCGATGATTCACTGAATTCTGCAATTCACAT
TACTTATCGCATTTCGCTGCGTTCTTCATCGATGCGAGAGCCAAGAGAT
CCGTTGTTGAAAGTTTTATTTTGTTATAAAATTTAATACATTCATAGAC
TTTGTGTTTATAAGTGAAATAGGAGTTCGCCCTCTTGCGAGAGTTACTA
TCCCAAACAAATGCACAGGGTTAGAAAGTGAGAGTTCGGACTCCAAGTT
AAGTTGGACGTCCTATATTCACTAATGATCCTTCCGCAGGTTCACCTAC
GGAAACCTTGTTACGACTTTTACTNNNNN

The nucleotide base symbols as used in the sequence listing are in accordance with the WIPO Standard ST.26 (Handbook of Industrial Property Information and Documentation—Recommended standard for the presentation of nucleotide and amino acid sequence listing using XML). Sequences may include the symbol “N”, representing an unknown nucleotide. The nucleotide represented by “N” at each location could be “A”, “T/U”, “C”, or

Flasks containing acidic liquid media having a pH of 3 and the pH indicator bromocresol purple were provided. The composition of the media was as follows:

500 mL of culture medium comprising 5 g of yeast extract, 3% (vol/vol) glycerol, 15 mM CaCl₂, 10 g of agar and 0.01% (wt/vol) of bromocresol purple was prepared with the balance being water. The pH of the medium was then adjusted to 3. After sterilization in an autoclave, 2.5 mL of ampicillin (stock 50 μg/ml) was added to the final medium.

The above-mentioned strains were added individually to each of the flasks. The flasks were then stoppered and maintained in aerobic conditions at room temperature ˜23° C. for 72 hours. After 24 hours, the pH of each of the liquid media in each of the flasks had increased to pH 7, thus demonstrating the ability of each of the yeast strains to rapidly increase the pH of those liquid media.

### Example 2—Viability of Yeasts in Acid Mine Drainage

A simulated AMD was prepared having the following composition:

| | Total | Molecular | | | |
|---|---|---|---|---|---|
| | Molecular | Weight of | Concentration | | |
| Compound | Weight | each element | (mg/L) | Added (mg) per 1 L: | |
| SO₄ | | 96 | | | |
| Al | 342 | 27 | 260 | AlSO₄ | 3,293 |
| Cd | 208 | 112 | 3.3 | CdSO₄ | 6 |
| Cu | 159 | 64 | 100 | CuSO₄ | 248 |
| Fe | 152 | 56 | 220 | FeSO₄ | 597 |
| Ni | 155 | 59 | 0.24 | NiSO₄ | 0.6 |
| Pb | 303 | 207 | 0.057 | PbSO₄ | 0.1 |
| Zn | 161 | 65 | 1,100 | ZnSO₄ | 2,724 |

The pH of the simulated AMD was adjusted to 3.0 using sulfuric acid. Flasks containing 20 ml of the simulated AMD were inoculated with 500 μL (final OD˜6) of strains Debaryomyces hansenii or Rhodotorula mucilaginosa. The samples were maintained at room temperature under aerobic conditions and were shaken at 200 rpm. The samples were exposed to light over a cycle of 8 hours of light and 16 hours of darkness.

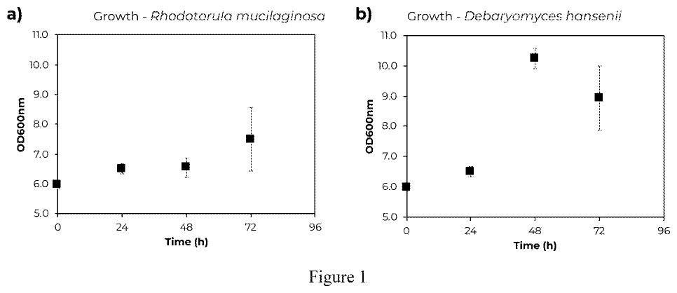

Samples were taken from each flask and analyzed for fungal growth (by measuring optical density of the sample using a DR3900 spectrophotometer (Hach, USA) and pH at 0 h, 24 h, 48 h and 72 h. The results are shown in FIG. 1 which demonstrate that the growth of both fungi continued during the course of the experiment, in spite of the low pH and dissolved metal content of the simulated AMD and that, surprisingly, the fungal populations are capable not only of surviving in such environments, but expanding.

### Example 3— Ability of Yeast to Increase pH and Reduce Metal Concentration of Acid Mine Drainage

An array of 6-well multiwell plates was used as an aerobic test system at 30 degrees Celsius and 100 RPM using two dilutions (0.2×, 1×) of Hestrin-Schramm media (Biochemical Journal 58:345-352, 1954). The starting pH was adjusted to 3.0 using sterile dilute H2504. Media in each well was then modified with target, varying concentrations (1×, 3× and 5×) of individual metals from standards in sulfuric acid as shown in the following table. Bromocresol green as a pH indicator was added to the media which were then inoculated with Debaryomyces hansenii. Solution pH measurements and content of three metals (aluminum, cadmium and iron) of the liquid samples were taken in duplicate on Day 14, averaged and the results observed are shown in the following table:

| | ICP Initial | | | Removal efficiency (%) | | | | | | |
|---|---|---|---|---|---|---|---|---|---|---|
| | Concentrations | | | (starting concentration vs. final | | | | | | |
| Selected | (mg/L) | | | concentration) | | | | Final pH | | |
| metals | 1x | 3x | 5x | 1x | 3x | 5x | Initial pH | 1x | 3x | 5x |
| Al | 278 | 883 | 1387 | 0%. | 75% | <10% | 3.0 | 8.0 | 5.0 | 3.0 |
| Cd | 6.8 | 20.4 | 34.1 | <10% | 15% | 15% | 3.0 | 8.0 | 8.8 | 8.5 |
| Fe | 189 | 568 | 946 | NA | 0% | 50% | 3.0 | 9.0 | 8.5 | 8.3 |

The precipitation of metal from the acidified medium, particularly iron, was also observed.

This data demonstrates that the methods of the present application advantageously result in the effective increase in the pH of simulated AMD as well as the removal of metals therefrom, demonstrating their effectiveness in the remediation of AMD and other acidic liquid media such as ARD and other types of wastewaters. The data also demonstrate that the pH raising and/or metal removal properties of fungi may vary at differing pH and/or metal concentrations, enabling one skilled in the art to optimize remediation processes by modifying pH and/or metal concentration.

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence, which is effective to meet the objectives there intended, unless the context specifically indicates the contrary.

1. A bioremediation method, comprising:
providing an alkalinizing acidophilic fungus,
contacting an acidic liquid having a pH of 5 or lower with the alkalinizing acidophilic fungus, and
maintaining the acidic liquid under conditions sufficient to permit the alkalinizing acidophilic fungus to increase the pH of the acidic liquid.

2. The method of claim 1, wherein the fungus is a yeast.

3. The method of claim 1, wherein the acidic liquid has a pH of 3 or lower.

4. The method of claim 1, wherein the acidic liquid comprises sulphate ions.

5. The method of claim 4, wherein the sulphate ions ae present at a concentration ranging from 0.1 to 20 grams per liter of the acidic liquid.

6. The method of claim 1, wherein the acidic liquid comprises dissolved heavy metal/'s.

7. The method of claim 6, wherein the dissolved heavy metals are present at a concentration of equal to or greater than about 500 mg per liter of the acidic liquid.

8. The method of claim 6, wherein the acidic liquid comprises one or more of dissolved copper, iron, nickel, cadmium, strontium, mercury, lead, arsenic, aluminum, lithium, zinc and/or manganese.

9. The method of claim 6, wherein the process results in the precipitation of dissolved heavy metals in the form of precipitated heavy metal's.

10. The method of claim 9, further comprising the step of collecting the precipitated heavy metal/s.

11. The method of claim 1, wherein the acidic liquid has a volume of about 10,000 liters or more.

12. The method of claim 1, wherein the acidic liquid is not buffered and/or wherein the pH of the acidic liquid is not adjusted prior to the addition of the fungus thereto.

13. The method of claim 1, wherein the acidic liquid is wastewater, optionally AMI).

14. The method of claim 1, wherein the fungus comprises a single strain of alkalinizing fungus.

15. The method of claim 14, wherein the alkalinizing fungus is a yeast or a mold.

16. The method of claim 1, wherein the fungus comprises a plurality of strains of alkalinizing fungus.

17. The method of claim 16, wherein the composition comprises one or more strains of alkalinizing yeast.

18. The method of claim 16, wherein the composition comprises one or more strains of alkalinizing mold.

19. The method of claim 1, further comprising contacting the acidic liquid with a plurality of compositions each comprising one or more strains of alkalinizing fungus.

20. The method of claim 19, wherein at least one of said plurality of compositions comprises one or more strains of alkalinizing yeast.

21. The method of claim 19, wherein at least one of said plurality of compositions comprises one or more strains of alkalinizing mold.

22. The method of claim 1, comprising adding a nutrition source to the acidic liquid.

23. The method of claim 22, wherein the nutrition source is a nitrogen source, optionally wherein the nitrogen source is soybean residues, effluent from the dairy industry, or an amino acid and/or protein-rich wastewater.

24. The method of claim 22, wherein the nutrition source is a carbon source, optionally wherein the carbon source is molasses.

25. The method of claim 22, wherein the nutrition source is comprised in a composition comprising the fungus.

26. The method of claim 22, further comprising adding a second nutrition source to the acidic liquid.

27. The method of claim 1, wherein the fungus is native.

28. The method of claim 1, wherein the fungus is heavy metal resistant.

29. The method of claim 28, wherein the fungus, when contacted with an acidic liquid medium comprising cadmium at a level of 2 mg per liter, copper at a level of 100 mg per liter, lead at a level of 0.05 mg per liter, iron at a level of 200 mg per liter, nickel at a level of per liter and or zinc at a level of 1000 mg per liter, retains its alkalinizing ability for 24 hours or longer.

30. The method of claim 1, wherein the fungus does not produce hydrogen sulfide.

31. The method of claim 1, wherein the fungus does not adsorb and/or sequester dissolved heavy metals.

32. The method of claim 1, wherein the process is conducted in a bioreactor.

33. The method of claim 1, wherein the process is conducted in situ.

34. The method of claim 1, wherein the pH of the acidic liquid is increased by at least 1 pH unit.

35. The method of claim 1, wherein the pH of the acidic liquid is increased by at least about 3 pH units.

36. The method of claim 1, wherein the pH of the acidic liquid is increased to neutral pH.

37. A kit comprising a composition comprising an alkalinizing acidophilic fungus and instructions to use the composition in a method of claim 1.
